(19) United States
(12) Patent Application Publication
(10) Pub. No.: US 2012/0029015 A1
(43) Pub. Date: Feb. 2, 2012

(54) Novel medical use of kynurenic acid, its precursors and derivatives

(76) Inventors: Andrzej Marciniak, Lublin (PL); Waldemar Turski, Lublin (PL)

(21) Appl. No.: 13/258,654

(22) PCT Filed: Mar. 23, 2010
(86) PCT No.: PCT/PL2010/050012

§ 371 Date: Sep. 22, 2011

(30) Foreign Application Priority Data
Mar. 23, 2009 (PL) P 387584

(51) Int. Cl. A61K 31/47; A61P 1/18; C07D 215/48

(52) U.S. Cl. 514/312; 546/156

(57) ABSTRACT

The invention relates to the use of kynurenic acid derivatives of general formula (I),

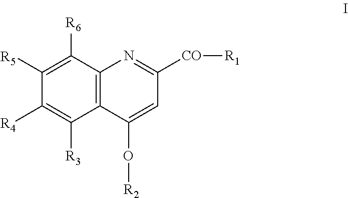

wherein
R¹ is hydroxy, NHR², NR²R² or C₁₋₁₀ straight or branched alkoxy or glyceryl group;
R² is hydrogen atom or C₁₋₁₀ straight or branched alkyl group;
R³-R⁶ are independently of each other hydrogen atom, halogen atom, C₁₋₁₀ alkyl, C₂₋₁₀ alkenyl or alkynyl group optionally substituted with a halogen atom,
and a pharmaceutically acceptable salt, ester or amide thereof
in manufacturing of a drug used in prevention and therapy of pancreas disorders characterized by edema and/or vacuolization and/or destruction of pancreatic tissue and/or pancreas necrosis leading to release of pancreatic α-amylase.

Preferably, in the compound represented by formula (I)
R¹ is a hydroxy, NH₂, NH—(C₁₋₅ straight or branched alkyl) or C₁₋₅ straight or branched-alkoxy or glyceryl group;
R² is a hydrogen atom;
R³-R⁶ are independently of each other a hydrogen atom, halogen atom or CF₃ group.

In the most preferred embodiment, the invention concerns the use of kynurenic acid or its pharmaceutically acceptable salt in manufacturing of a drug used in prevention and therapy of pancreas disorders characterized by edema and/or vacuolization and/or destruction of pancreatic tissue and/or pancreas necrosis leading to release of pancreatic α-amylase.

### CROSS-REFERENCE TO RELATED APPLICATION

This application is a Section 371 National Stage Application of International Application No. PCT/PL2010/050012, filed Marc 23, 2010 and published as WO 2010/110689 A1 on Sep. 30, 2010, the content of which is hereby incorporated by reference in its entirety.

### TECHNICAL FIELD

The subject of the invention is the use of kynurenic acid, its precursors and derivatives and physiologically acceptable salts in the manufacture of a medicament used for the treatment of and prevention of conditions characterized by edema and/or vacuolization and/or destruction of pancreatic tissue leading to release of pancreatic α-amylase.

### BACKGROUND ART

Pancreas disorders represent a significant therapeutic issue in healthcare. Pancreatic diseases are disorders characterized by destruction of pancreatic tissue accompanied by decrease of the exocrine and endocrine function of pancreas. Serious multi-organ failure may occur during pancreas disorders. Pancreatic tissue disorder is one of a few disorders in which mortality has not changed over decades and the morbidity remains high.

Nowadays only symptomatic or palliative treatment is available, whose effectiveness is limited. It includes: pain control, fluid and electrolyte management, oxygen supplementation, nasogastric suction, mechanical ventilation, administration of inotropes and antibiotics, or surgery. Therefore, there is a need for new effective drugs.

In human and mouse pancreas, presence of various receptors including GPR35 receptors has been confirmed (Wang et al., 2006; J Biol Chem 281; 22021-22028). In pancreas, glutamatergic receptors and specific glutamate transporters have been described (Hayashi et al., J Histochem Cytochem 2003; 51: 1375-1390). Nicotinergic receptors have also been confirmed in pancreas (Kirchgessner and Liu, J Comp Neurol 1998; 390: 497-514; Love, Auton Neurosci 2000; 84: 68-77; Rayford and Chowdhury, J Assoc Acad Minor Phys 2001; 12: 105-108).

On the other hand kynurenic acid is an endogenous agent present in various tissues and fluids in the mammalian body. Concentration of kynurenic acid measured in different tissues and fluids in humans and other mammals varies from 0.04 to 16 μM (Urbanska 2006, Pharmacol Rep, 58, 507-511; Kuc et al., 2008, Amino Acids, 35, 503-505). It has been demonstrated that kynurenic acid mitigates development of oxidative stress by means of prevention of lipid peroxidation (Kaszaki et al., Neurogastroenterol Motil 2008; 20: 53-62). Kynurenic acid also possesses analgesic activity (Näsström et al., Eur J Pharmacol 1992; 212: 21-9). It is also known that kynurenic acid protects against gastroduodenal ulceration (Glavin i wsp., Prog Neuropsychopharmacol Biol Psychiatry 1989; 13: 569-72, Res Commun Chem Pathol Pharmacol 1989; 64: 111-9). Kynurenic acid has been recently demonstrated to be active in gastroenterological conditions characterized by increased peristaltic activity (Kaszaki et al., Neurogastroenterol Motil 2008; 20: 53-62; WO 2008/087461). Kynurenic acid is also active in therapy of sepsis (WO/2006/117624).

Experiments showed that kynurenic acid interacts with GPR35 receptors (Wang et al., 2006; J Biol Chem, 281, 22021-22028). Kynurenic acid is also an antagonist of α7 nicotinic receptor (Hilmas et al., 2001, J Neurosci, 21, 7463-7473). In addition, kynurenic acid interacts with other receptors, such as glutamatergic N-methyl-D-aspartate (NMDA) and α-amino-3-hydroxy-5-methyl-4-isoxazolopropionate (AMPA) receptors (Kemp et al., 1988, Proc Natl Acad Sci USA, 85, 6547-6550, Hilmas et al., 2001, J Neurosci, 21, 7463-7473).

### SUMMARY OF INVENTION

### Technical Problem

Contemporary therapy of pancreas disorders leading to destruction of pancreatic tissue should consist of two components:

Decrease of exocrine pancreas function in order to restrict self destruction of pancreas;

Restriction of several processes (including inhibiting free-radical processes) leading to auto destruction and multi-organ complications.

The aim of the invention is to provide substances suitable for the manufacture of a modern medicament for the treatment and prevention of pancreatic disorders characterized by edema and/or vacuolization and/or destruction of pancreatic tissue leading to release of pancreatic α-amylase.

### Solution to Problem

It was found that kynurenic acid possesses properties which are useful when manufacturing a medicament for the treatment and prevention of conditions characterized by edema and/or vacuolization and/or destruction of pancreatic tissue leading to release of pancreatic α-amylase. Moreover, similar effects are exerted by derivatives of kynurenic acid.

The invention relates to the use of kynurenic acid derivatives of general formula (I),

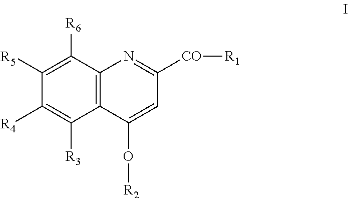

wherein
R¹ is hydroxy, NHR², NR²R² or C₁₋₁₀ straight or branched alkoxy or glyceryl group;
R² is hydrogen atom or C₁₋₁₀ straight or branched alkyl group;
R³-R⁶ are independently of each other hydrogen atom, halogen atom, C₁₋₁₀ alkyl, C₂₋₁₀ alkenyl or alkynyl group optionally substituted with a halogen atom,
and a pharmaceutically acceptable salt, ester or amide thereof
in manufacturing of a drug used in prevention and therapy of pancreas disorders characterized by edema and/or vacuolization and/or destruction of pancreatic tissue and/or pancreas necrosis leading to release of pancreatic α-amylase.
Preferably, in the compound represented by formula (I)
R¹ is a hydroxy, NH₂, NH—(C₁₋₅ straight or branched alkyl) or C₁₋₅ straight or branched-alkoxy or glyceryl group;
R² is a hydrogen atom;
R³-R⁶ are independently of each other a hydrogen atom, halogen atom or CF₃ group.

In the most preferred embodiment, the invention concerns the use of kynurenic acid represented by a chemical formula

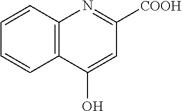

or its pharmaceutically acceptable salt in manufacturing of a drug used in prevention and therapy of pancreas disorders characterized by edema and/or vacuolization and/or destruction of pancreatic tissue and/or pancreas necrosis leading to release of pancreatic α-amylase.

Conditions promoting pancreas disorders characterized by edema and/or vacuolization and/or destruction of pancreatic tissue leading to release of pancreatic α-amylase may occur in diseases comprising: gallstones or gallbladder disease; injury to the abdomen; viral infections including mumps, smallpox, measles, rubella, mononucleosis, hepatitis, and other evoked by viruses Echo, Coxsackie, HIV; bacterial infections including typhus, diphtheria, leptospirosis, toxoplasmosis, Bang's disease, syphilis, tuberculosis, scarlet fever; fungal diseases including Aspergillus; autoimmune disorders; parasitic diseases including human ascaris infection; mucoviscidosis; hypertriglyceridemia; hyperchylomicronemia; obesity; hyperparathyroidism; other diseases with elevated calcium level such as vitamin D intoxication, sarcoidosis, Vater's diverticulum, duodenal stenosis; pathological conditions caused by ethanol ingestion, malnutrition; steatorrhoea; coeliac disease; food allergy; ischemia, trauma related to endoscopic retrograde cholangiopancreatography or sphincter of Oddi manometry; trauma due to diagnostic or surgical procedures concerning the pancreas or gallbladder and biliary tract including edema or hematoma induced by endoscopic papillotomy; surgery on spleen or heart including cardiac transplantation; pancreas dysfunction due to medications such as for example: steroids, chemotherapeutic agents, hydrochlorothiazide, furosemide, estrogen, tetracyclines, sulfonamides, barbiturates, valproic acid, capoten; pancreas dysfunction due to carbon monoxide poisoning, methyl alcohol intoxication or scorpion sting.

The invention refers also to the use of substances leading to increase of kynurenic acid concentration in a body for manufacturing of a drug used in prevention and therapy of pancreas disorders characterized by edema and/or vacuolization and/or destruction of pancreatic tissue and/or pancreas necrosis leading to release of pancreatic α-amylase. Enhancement of kynurenic acid concentration can be induced by substances either increasing the activity of enzymes synthesizing kynurenic acid or inhibiting kynurenine metabolism. It is thus possible to increase the kynurenic acid concentration by enhacement of kynurenine aminotransferase activity or by inhibition of kynurenine metabolism initiated by kynureninase or kynurenine 3-hydroxylase, e.g. by nitrobenzoyl-alanine or nicotinyl-alanine.

Enhancement of kynurenic acid concentration can be induced also by substances leading to augmentation of production of kynurenic acid e.g. due to increased cellular energy metabolism, or pyridoxal 5′-phosphate, or L-amino acids, or alpha-keto acids, or changes in the ionic milieu. Enhancement of kynurenic acid concentration can be also induced by inhibition of its excretion, e.g. by combination of kynurenic acid and probenecid.

Preferably, substances leading to enhanced production of kynurenic acid are precursors of kynurenic acid, e.g. kynurenine, indole-3-pyruvate.

The invention describes the method of prevention and treatment of conditions characterized by edema or vacuolization or destruction of pancreatic tissue leading to release of pancreatic α-amylase, in which a therapeutically effective amount of kynurenic acid derivative of formula (I) and substituents as described above is administered to a patient. The method preferably comprises administration of a therapeutically effective amount of kynurenic acid or a pharmaceutically acceptable salt, ester or amide thereof to the body.

The invention also relates to the method of prevention and treatment of conditions characterized by edema or vacuolization or destruction of pancreatic tissue leading to release of pancreatic α-amylase comprising administration of a therapeutically effective amount of substances leading to increase of kynurenic acid concentration in the body, such as kynurenic acid precursor kynurenine or indole-3-pyruvate.

The compounds represented by general formula (I) can be produced by methods well known to the person skilled in the art.

It is to be understood that in any of the embodiments described herein, salts of high solubility are particularly preferable for medical use. The pharmaceutically acceptable salts can be formed with inorganic acids such as hydrohalic acids, including hydrochloric or hydrobromic acid, phosphoric acid, metaphosphoric acid, nitric acid or sulphuric acid, or with organic acids like acetic acid, benzosulfonic acid, benzoic acid, citric acid, ethanedulfonic acid, fumaric acid, gluconic acid, glycolic acid, isothonic acid, lactic acid, lactobionic acid, maleic acid, malic acid, succinic acid, p-toluenesulfonic acid, tartaric acid and the like acids.

Basic salts appropriate for pharmaceutical use can be ammonium salts, alkali metal salts (like sodium or potassium), alkali earth metal salts (like calcium or magnesium), and amine salts.

Kynurenic acid, its salts and derivatives to be used for the manufacturing of a medication preventing or treating pancreas disorders can be administered parenterally (i.e. intravenously, intramuscularly, subcutaneously, intracavally), enterally (i.e. per os, gatrically, duodenally, jejunally, rectally), by means of inhalation or transdermally. The necessary dose depends on chemical properties of the compound used, the form of the drug, the route of administration, age and condition of the patient. According to own investigations, it was shown that administration of kynurenic acid intravenously in the dose of 50 or 100 mg/kg/hr in rats was well tolerated.

In the illustrative embodiment of the methods described herein, kynurenic acid or pharmaceutically acceptable salts thereof or kynurenic acid derivative, or kynurenic acid precursor described herein is illustratively administered to a patient orally in the range from about 0.1 mg/kg to about 1 g/kg, from about 0.1 mg/kg to about 1 mg/kg, from about 0.1 mg/kg to about 10 mg/kg, from about 1 mg/kg to about 10 mg/kg, from about 1 mg/kg to about 100 mg/kg, from about 1 mg/kg to about 200 mg/kg, from about 1 mg/kg to about 500 mg/kg, from about 1 mg/kg to about 1 g/kg, from about 10 mg/kg to about 100 mg/kg, from about 10 mg/kg to about 200 mg/kg, from about 10 mg/kg to about 500 mg/kg, from about 10 mg/kg to about 1 g/kg, from about 20 mg/kg to about 50 mg/kg, from about 20 mg/kg to about 100 mg/kg, from about 20 mg/kg to about 200 mg/kg, from about 50 mg/kg to about 100 mg/kg, from about 50 mg/kg to about 200 mg/kg, from about 100 mg/kg to about 200 mg/kg, from about 100 mg/kg to about 500 mg/kg, or from about 100 mg/kg to about 1 g/kg, In another illustrative embodiment of the methods described herein, kynurenic acid or pharmaceutically acceptable salts thereof or kynurenic acid derivative, or kynurenic acid precursor described herein is illustratively administered to a patient parenterally in the range from about 0.1 mg/kg to about 1 g/kg, from about 0.1 mg/kg to about 1 mg/kg, from about 0.1 mg/kg to about 10 mg/kg, from about 1 mg/kg to about 10 mg/kg, from about 1 mg/kg to about 100 mg/kg, from about 1 mg/kg to about 200 mg/kg, from about 1 mg/kg to about 500 mg/kg, from about 1 mg/kg to about 1 g/kg, from about 10 mg/kg to about 100 mg/kg, from about 10 mg/kg to about 200 mg/kg, from about 10 mg/kg to about 500 mg/kg, from about 10 mg/kg to about 1 g/kg, from about 20 mg/kg to about 50 mg/kg, from about 20 mg/kg to about 100 mg/kg, from about 20 mg/kg to about 200 mg/kg, from about 50 mg/kg to about 100 mg/kg, from about 50 mg/kg to about 200 mg/kg, from about 100 mg/kg to about 200 mg/kg, from about 100 mg/kg to about 500 mg/kg, or from about 100 mg/kg to about 1 g/kg,

In another embodiment, when used kynurenic acid or pharmaceutically acceptable salts thereof or kynurenic acid derivative, or kynurenic acid precursor, are administered orally to a patient at a daily dose of 0.1, 0.5, 1.0, 2.0, or 5.0 mg/kg, corresponding to approximately, 7.0, 35, 70, 140, or 350 mg for an average weight adult. Without limiting the foregoing, it is appreciated that such lower doses may be more applicable to an ongoing, or chronic therapy, designed for continuous administration, rather than intermittent or acute administration. Accordingly, the daily dose may be divided and administered b.i.d. and/or t.i.d, although it is to be understood that q.d. dosing is described herein. It is to be understood that the illustrative doses described herein represent daily doses, and may be therefore administered q.d., b.i.d., t.i.d., and according to additional dosing protocols. In addition, it is to be understood that the doses may be single or divided.

### Advantageous Effects of Invention

Our experiments demonstrated that kynurenic acid, which interacts with GPR35, glutamatergic and α7-nicotinic receptors, induces decrease of exocrine pancreas function, restriction of self destruction of pancreas and restriction of several processes (including inhibiting free-radical processes) leading to auto destruction and multi-organ complications, thus protecting against homeostatic dysregulation.

The invention provides the method of affecting these receptors by administration of kynurenic acid or its derivatives, which is effective in the treatment or preventive treatment of conditions characterized by edema or vacuolization or destruction of pancreatic tissue leading to release of pancreatic α-amylase. According to the invention, the substances described induce decrease of exocrine pancreas function, restriction of self destruction of pancreas and restriction of several processes (including inhibiting free-radical processes) leading to auto destruction and multi-organ complications, thus protecting against homeostatic dysregulation.

### DESCRIPTION OF EMBODIMENTS

### Methodology

The experiments were performed using adult male Wistar rats. All experimental procedures were in compliance with currently accepted guidelines for care and use of animals. The study protocol was approved by the Bioethics Committee. Before the experiment, rats were given no food for 24 hours, water was available in unrestricted quantities. Destruction of pancreatic tissue (DPT) was induced by injections of caerulein (dose of 15 μg/kg/hr). Animals were randomly assigned to the following groups. Group 1 (Control—control group): animals received only saline pretreatment and saline infusion (1 ml/kg/hr), group 2 (DPT): animals received caerulein and the saline infusion (1 ml/kg/hr) and group 3 (DPT+KYNA): animals received caerulein and infusion of buffered solution of kynurenic acid in the dose of 50 or 100 mg/kg/hr. After 6 hours from inducing DPT antioxidative effects were determined by means of measuring the ferric reducing ability of plasma (FRAP) and levels of the markers of oxidative stress: malonyldialdehyde and 4-hydroxynonenals (MDA+4-HNE). The measurement of pancreatic edema, the serum α-amylase activity and histopathological analysis of pancreas were carried out to assess the progress of the disease.

### Example 1

### Water Content of Pancreatic Tissue (% of Wet Weight)

TABLE 1
Water content of pancreas.

| | Water content [%] | Standard deviation (±SD) |
|---|---|---|
| Control | 64.54 | 2.83 |
| DPT | 87.58^{#} | 2.57 |
| KYNA 50 + DPT | 73.03* | 4.99 |
| KYNA 100 + DPT | 68.22* | 6.70 |

Results represent means ± SD values;
^{#}p < 0.05 vs. Control;
*p < 0.05 vs. DPT group (one way ANOVA).
Control, control group;
DPT, destruction of pancreatic tissue;
KYNA, kynurenic acid; KYNA was administered in the dose of 50 or 100 mg/kg/hr.

A prominent feature of destruction of pancreatic tissue on macroscopic presentation is edema, which was seen in the group of animals subjected to caerulein. On the contrary, in the group of animals receiving caerulein and an infusion of kynurenic acid in the dose of 100 mg/kg/hr (DPT+KYNA) no edema was noted. In addition, a significantly lower degree of water content of pancreas was noted in this group (DPT+KYNA) in comparison with the group of untreated animals with destruction of pancreatic tissue (DPT). Kynurenic acid in the dose of 50 mg/kg/hrs was also effective (Table 1). Since kynurenic acid prevents edema induced by the pancreas damaging factor caerulein, kynurenic acid, its salts and derivatives may be used in prevention and therapy of conditions leading to injury of pancreatic tissue.

### Example 2

### Histological Changes in Pancreatic Tissue

TABLE 2
Histological changes in pancreatic tissue.

| | Edema | Vacuolisation | Necrosis |
|---|---|---|---|
| | (0-3) | (0-3) | (0-3) |
| Control | 0 | 0 | 0 |
| DPT | 2.6 ± 0.57 | 3.0 ± 0.00 | 0.6 ± 0.52 |
| KYNA + DPT | 1.6 ± 0.45* | 0.8 ± 0.44* | 0.0 ± 0.0* |

Results represent means ± SD values,
*p < 0.05 vs. DPT group (one way ANOVA).
Control, control group;
DPT, destruction of pancreatic tissue;
KYNA, kynurenic acid. KYNA was administered in the dose of 100 mg/kg/hr.

Histopathological presentation of pancreas injury consists of edema, vacuolization or necrosis (Table 2). Since in the situation of application of a pancreas damaging agent caerulein kynurenic acid in the dose of 100 mg/kg/hr prevents or diminishes edema, vacuolization, and number of necrotic foci, kynurenic acid, its salts and derivatives may be of use in prevention and therapy of conditions leading to destruction of pancreatic tissue and are characterized by such histological picture.

### Example 3

### Serum α-Amylase Activity

TABLE 3
Serum α-amylase activity.

| | Amylase activity [U/l] | Standard deviation (±SD) |
|---|---|---|
| Control | 2559.72 | 470.60 |
| DPT | 10601.25^{#} | 1579.45 |
| KYNA 50 + DPT | 5262.26^{#}* | 829.99 |
| KYNA 100 + DPT | 3495.20* | 326.43 |

Results represent means ± SD values;
^{#}p < 0.05 vs. Control;
*p < 0.05 vs. DPT group (one way ANOVA).
Control, control group;
DPT, destruction of pancreatic tissue;
KYNA, kynurenic acid. KYNA was administered in the dose of 50 or 100 mg/kg/hr.

The extent of the serum α-amylase elevation is roughly proportional to the degree of destruction of pancreatic tissue. In Table 3 it is shown that kynurenic acid in the dose of 100 mg/kg/hr prevents elevation of α-amylase in the serum of caerulein-treated rats. Kynurenic acid in the dose of 50 mg/kg/hrs was also effective. Since in the case of action of an agent damaging pancreatic tissue kynurenic acid prevents elevation of α-amylase activity in plasma, kynurenic acid, its salts and derivatives may be of use in prevention and therapy of conditions leading to the damage of pancreatic tissue.

### Example 4

### Plasma Levels of Lipid Peroxidation Products

TABLE 4
Plasma levels of lipid peroxidation products: malonyldialdehyde
and 4-hydroxynonenals (MDA + 4-HNE).

| | MDA + 4-HNE [nmol/ml] | Standard deviation (±SD) |
|---|---|---|
| Control | 3.24 | 0.44 |
| DPT | 4.72^{#} | 0.39 |
| KYNA + DPT | 3.11* | 0.32 |

Results represent means ± SD values;
^{#}p < 0.05 vs. Control;
*p < 0.05 vs. DPT group (one way ANOVA).
Control, control group;
DPT, destruction of pancreatic tissue;
KYNA, kynurenic acid. KYNA was administered in the dose of 100 mg/kg/hr.

In the group of animals with caerulein-induced destruction of pancreatic tissue significant increase of concentration of malonyldialdehyde (MDA) and 4-hydroxynonenals (4-HNE), two markers of oxidative stress, occurred. As shown in Table 4, in groups of animals subjected to kynurenic acid (100 mg/kg/hr), concentrations of MDA and 4-HNE were low and did not significantly differ from control. These findings suggests that kynurenic acid may prevent lipid peroxidation and destructive processes in pancreas disorders leading to the destruction of the pancreatic tissue.

### Example 5

### The Antioxidant Capacity of Plasma

TABLE 5
Antioxidant capacity of plasma
(FRAP—Ferric Reducing Ability of Plasma)

| | FRAP (μM) | Standard deviation (±SD) |
|---|---|---|
| Control | 400.0 | 25.4 |
| DPT | 358.1 | 34.0 |
| KYNA + DPT | 602.4* | 182.2 |

Results represent means ± SD values;
*p < 0.05 vs. DPT group (one way ANOVA).
Control, control group;
DPT, destruction of pancreatic tissue;
KYNA, kynurenic acid. KYNA was administered in the dose of 100 mg/kg/hr.

In the group of animals subjected to caerulein-induced destruction of pancreatic tissue, the ferric reducing ability of plasma (FRAP) did not differ compared to control group, as shown in Table 5. However, administration of kynurenic acid (100 mg/kg/hrs) increased significantly the elements of antioxidative defence FRAP. This finding indicates that kynurenic acid, its salts and derivatives may improve antioxidative defence and thus be of therapeutic value in pancreas disorders leading to the destruction of the pancreatic tissue.

Since kynurenic acid is active on GPR35 receptors, NMDA receptors and nicotinic α-7 receptors, drugs affecting these receptors alone or in combination e.g. GPR35 receptor, NMDA receptor and nicotinic α-7 receptor; GPR35 receptor and NMDA receptor; GPR35 receptor and nicotinic α-7 receptor; and NMDA receptor and nicotinic α-7 receptor may influence pancreas disorders in a similar way to kynurenic acid.

Although the present invention has been described with reference to preferred embodiments, workers skilled in the art will recognize that changes may be made in form and detail without departing from the spirit and scope of the invention.

1. Kynurenic acid derivatives of general formula (I), wherein 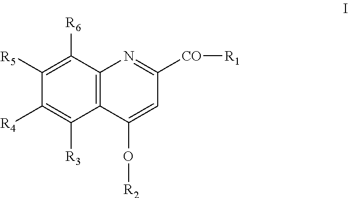
R¹ is a hydroxy, NHR², NR²R² or C₁₋₁₀ straight or branched alkoxy or glyceryl group;
R² is a hydrogen atom or a C₁₋₁₀ straight or branched alkyl group;
R³-R⁶ are independently of each other hydrogen atom, halogen atom, C₁₋₁₀ alkyl, C₂₋₁₀ alkenyl or alkynyl group optionally substituted with a halogen atom,
or a pharmaceutically acceptable salt, ester or amide thereof,
in manufacturing of a drug used in prevention and therapy of pancreas disorders characterized by edema and/or vacuolization and/or destruction of pancreatic tissue and/or pancreas necrosis leading to release of pancreatic α-amylase.

2. The kynurenic acid derivatives according to claim 1, wherein
R¹ is hydroxy, NH₂, NH—(C₁₋₅ straight or branched alkyl) or C₁₋₅ straight or branched-alkoxy or glyceryl group;
R² is a hydrogen atom;
R³-R⁶ are independently a hydrogen atom, halogen atom or CF₃ group.

3. The kynurenic acid derivatives according to claim 1, wherein the derivative is kynurenic acid of general formula 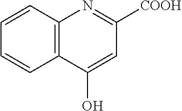
or pharmaceutically acceptable salt thereof.

4. The kynurenic acid derivatives according to claim 1 in conditions promoting pancreas disorders characterized by edema or vacuolization or destruction of pancreatic tissue leading to release of pancreatic α-amylase occurring in the following diseases: gallstones or gallbladder disease; injury to the abdomen; viral infections including mumps, smallpox, measles, rubella, mononucleosis, hepatitis, and other evoked by viruses Echo, Coxsackie, HIV; bacterial infections including typhus, diphtheria, leptospirosis, toxoplasmosis, Bang's disease, syphilis, tuberculosis, scarlet fever; fungal diseases including Aspergillus; autoimmune disorders; parasitic diseases including human ascaris infection; mucoviscidosis; hypertriglyceridemia; hyperchylomicronemia; obesity; hyperparathyroidism; other diseases with elevated calcium level such as vitamin D intoxication, sarcoidosis, Vater's diverticulum, duodenal stenosis; pathological conditions caused by ethanol ingestion), malnutrition; steatorrhoea; coeliac disease; food allergy; ischemia, trauma related to endoscopic retrograde cholangiopancreatography or sphincter of Oddi manometry; trauma due to diagnostic or surgical procedures concerning the pancreas or gallbladder and biliary tract including edema or hematoma induced by endoscopic papillotomy; surgery on spleen or heart including cardiac transplantation; pancreas dysfunction due to medications such as for example: steroids, chemotherapeutic agents, hydrochlorothiazide, furosemide, estrogen, tetracyclines, sulfonamides, barbiturates, valproic acid, capoten; pancreas dysfunction due to carbon monoxide poisoning, methyl alcohol intoxication or scorpion sting.

5. The kynurenic acid derivatives of a substance leading to increase of kynurenic acid concentration in a body in manufacturing of a drug used in prevention and therapy of pancreas disorders characterized by edema and/or vacuolization and/or destruction of pancreatic tissue and/or pancreas necrosis leading to release of pancreatic α-amylase.

6. The kynurenic acid derivatives according to claim 5, wherein the precursor of kynurenic acid is applied.

7. The kynurenic acid derivatives according to claim 5, wherein enhancement of kynurenic acid concentration is due to stimulation of kynurenic acid synthesis.

8. The kynurenic acid derivatives according to claim 5, wherein enhancement of kynurenic acid concentration is due to stimulation of activity of kynurenine aminotransferase.

9. The kynurenic acid derivatives according to claim 5, wherein enhancement of kynurenic acid concentration is due to inhibition of kynurenine metabolism at the level of kynurenine hydroxylase or kynureninase.

10. The kynurenic acid derivatives according to claim 5, wherein enhancement of kynurenic acid concentration is due to inhibition of kynurenic acid excretion.

11. The kynurenic acid derivatives according to claim 5 in conditions promoting pancreas disorders characterized by edema or vacuolization or destruction of pancreatic tissue leading to release of pancreatic α-amylase occurring in the following diseases: gallstones or gallbladder disease; injury to the abdomen; viral infections including mumps, smallpox, measles, rubella, mononucleosis, hepatitis, and other evoked by viruses Echo, Coxsackie, HIV; bacterial infections including typhus, diphtheria, leptospirosis, toxoplasmosis, Bang's disease, syphilis, tuberculosis, scarlet fever; fungal diseases including Aspergillus; autoimmune disorders; parasitic diseases including human ascaris infection; mucoviscidosis; hypethighlyceridemia; hyperchylomicronemia; obesity; hyperparathyroidism; other diseases with elevated calcium level such as vitamin D intoxication, sarcoidosis, Vater's diverticulum, duodenal stenosis; pathological conditions caused by ethanol ingestion, malnutrition; steatorrhoea; coeliac disease; food allergy; ischemia, trauma related to endoscopic retrograde cholangiopancreatography or sphincter of Oddi manometry; trauma due to diagnostic or surgical procedures concerning the pancreas or gallbladder and biliary tract including edema or hematoma induced by endoscopic papillotomy; surgery on spleen or heart including cardiac transplantation; pancreas dysfunction due to medications such as for example: steroids, chemotherapeutic agents, hydrochlorothiazide, furosemide, estrogen, tetracyclines, sulfonamides, barbiturates, valproic acid, capoten; pancreas dysfunction due to carbon monoxide poisoning, methyl alcohol intoxication or scorpion sting.

12. A method for treatment of pancreas disease characterized by edema and/or vacuolization and/or destruction of pancreatic tissue and/or pancreatic necrosis leading to release of pancreatic α-amylase, wherein therapeutically effective amount of kynurenic acid derivative is administered.

13. The method according to claim 12, wherein therapeutically effective amount of kynurenic acid or pharmaceutically acceptable salt, ester or amide thereof, is applied.

14. A method for treating condition or pancreas disease characterized by edema and/or vacuolization and/or destruction of pancreatic tissue and/or pancreatic necrosis leading to release of pancreatic α-amylase, wherein therapeutically effective amount of a substance evoking increase of kynurenic acid concentration is applied.

15. The method according to claim 14, wherein therapeutically effective amount of kynurenic acid precursor is applied.
